(19) United States
(12) Patent Application Publication
(10) Pub. No.: US 2013/0324927 A1
(43) Pub. Date: Dec. 5, 2013

(54) SYRINGE ASSEMBLY FOR A MEDICATION PUMP OR THE LIKE

(71) Applicants: Ramzi ABULHAJ, Charlotte, NC (US); Rick ADMANI, Charlotte, NC (US); Mark LINDSAY, Coventry, CT (US)

(72) Inventors: Ramzi ABULHAJ, Charlotte, NC (US); Rick ADMANI, Charlotte, NC (US); Mark LINDSAY, Coventry, CT (US)

(73) Assignee: CHOICE MEDICAL, LLC, Charlotte, NC (US)

(21) Appl. No.: 13/485,365

(22) Filed: May 31, 2012

(51) Int. Cl. A61M 5/145; B23P 19/04

(52) U.S. Cl. 604/151; 29/777

(57) ABSTRACT

The present invention provides a syringe assembly for a medication pump, such as an insulin pump, or the like that includes a pair of protruding structures at one end that mate with the typical pair of opposed recesses or other notch structures of the syringe assembly mating portion of the pump, without the use of a cap structure and needle member, and that may be manufactured primarily from a relatively inexpensive plastic material or the like, while still providing the medication supply back-pressure required to be adequately sensed by the pump electronics, such that the pump accepts the syringe assembly and is “primed” for proper operation.

### FIELD OF THE INVENTION

The present invention relates generally to a syringe assembly for a medication pump or the like. More specifically, the present invention relates to a syringe assembly for a medication pump or the like that includes an improved pump engagement portion and an improved back-pressure generation portion such that the pump, through its pump electronics, properly senses the syringe assembly and “primes” itself.

### BACKGROUND OF THE INVENTION

Insulin pumps are widely used by diabetics to selectively administer metered doses of insulin in the management of their conditions. These pumps consist of electronic devices that are coupled to infusion tube sets and/or devices that are operable for delivering insulin into the body of the user, on a timed schedule and/or on demand. The insulin is typically provided in a separate syringe assembly that is inserted into the pump. Preferably, these syringe assemblies are sealed and sterile prior to use.

Some of the pumps include a syringe assembly mating portion that includes a pair of opposed recesses or other notch structures that are configured to receive a corresponding pair of protruding structures, optionally including detents, associated with a cap structure that is disposed over an end of the syringe assembly after it is inserted into the pump. The interior of this cap structure includes a needle member that punctures a sealing membrane manufactured into the end of the syringe assembly. These needle members are typically made of a metal, such that their narrow interior diameter may be carefully controlled. This coupling of the cap structure to the syringe assembly mating portion of the pump secures the syringe assembly within the pump, and the needle member couples the infusion tube set and/or device to the supply of insulin. A piston member controlled by the pump electronics and acting on the insulin supply at the other end of the syringe assembly is operable for “pushing” the insulin out of the syringe assembly, through the needle member and infusion tube set and/or device, and into the body of the user, in a predetermined and controlled manner.

Often, the pump electronics are designed to “prime” the pump after a new syringe assembly is inserted (and/or an old syringe assembly is reinserted) into the pump, advancing the piston member to a predetermined degree. This is typically accomplished by the pump electronics sensing a certain amount of back-pressure, or resistance to expulsion, within the insulin supply as the piston member is advanced in a controlled manner.

Disadvantageously, with needle members that are made of plastic, or other plastic conduits that couple an insulin supply to an infusion tube set and/or device (with or without a cap structure), the narrow interior diameter required to create the proper back-pressure needed for effective “priming” may not be provided from a manufacturing standpoint, at least not cost effectively. In such cases, the pump electronics never sense the required back-pressure and the pump does not “prime,” potentially expelling all of the valuable insulin. Such pumps simply do not work with such syringe assemblies.

Thus, what is needed in the art is a syringe assembly for a medication pump, such as an insulin pump, or the like that includes a pair of protruding structures at one end that mate with the typical pair of opposed recesses or other notch structures of the syringe assembly mating portion of the pump, without the use of a cap structure and needle member, and that may be manufactured primarily from a relatively inexpensive plastic material or the like, while still providing the medication supply back-pressure required to be adequately sensed by the pump electronics, such that the pump accepts the syringe assembly and is “primed” for proper operation.

### BRIEF SUMMARY OF THE INVENTION

In various exemplary embodiments, the present invention provides a syringe assembly for a medication pump, such as an insulin pump, or the like that includes a pair of protruding structures at one end that mate with the typical pair of opposed recesses or other notch structures of the syringe assembly mating portion of the pump, without the use of a cap structure and needle member, and that may be manufactured primarily from a relatively inexpensive plastic material or the like, while still providing the medication supply back-pressure required to be adequately sensed by the pump electronics, such that the pump accepts the syringe assembly and is “primed” for proper operation.

In one exemplary embodiment, the present invention provides a syringe assembly for use in a medication pump or the like, including: a housing configured to selectively contain liquid medication or the like; a plunger member selectively disposed within the housing configured to selectively expel the liquid medication or the like from the housing through a tube connecting portion of the housing; and a piston member selectively coupled to the plunger member configured to selectively translate the plunger member within the housing; wherein the housing comprises a concentric handle structure and a plurality of angled fin structures configured to mate with a pair of opposed recesses or other notch structures of a syringe assembly mating portion of the medication pump or the like upon rotation; and wherein the housing comprises a flow restricting collar disposed therein to limit the flow of the liquid medication or the like from the housing, thereby creating a predetermined back-pressure upon translation of the plunger member within the housing. Optionally, the housing further comprises a plurality of protruding structures configured to mate with the pair of opposed recesses or other notch structures of the syringe assembly mating portion of the medication pump or the like upon rotation. The plurality of angled fin structures and the plurality of protruding structures of the housing are disposed on a neck portion of the housing substantially adjacent to the concentric handle structure of the housing. The plunger member further comprises one or more concentric sealing members disposed about the external periphery thereof configured to seal the liquid medication or the like within the housing. The plunger member further comprises a protruding portion with a sealed tapered end configured to fit inside tube connecting portion of the housing upon full deployment of the plunger member within the housing, thereby ensuring that all medication or the like is expelled. The tube connecting portion of the housing comprises a standard lure connection or the like. The flow restricting collar comprises a tapered portion. Optionally, the flow restricting collar is made of a metal or the like. Preferably, the flow restricting collar has a minimum interior diameter on the order of millimeters or less.

In another exemplary embodiment, the present invention provides a method for providing a syringe assembly for use in a medication pump or the like, comprising: providing a housing configured to selectively contain liquid medication or the like; providing a plunger member selectively disposed within the housing configured to selectively expel the liquid medication or the like from the housing through a tube connecting portion of the housing; and providing a piston member selectively coupled to the plunger member configured to selectively translate the plunger member within the housing; wherein the housing comprises a concentric handle structure and a plurality of angled fin structures configured to mate with a pair of opposed recesses or other notch structures of a syringe assembly mating portion of the medication pump or the like upon rotation; and wherein the housing comprises a flow restricting collar disposed therein to limit the flow of the liquid medication or the like from the housing, thereby creating a predetermined back-pressure upon translation of the plunger member within the housing. Optionally, the housing further comprises a plurality of protruding structures configured to mate with the pair of opposed recesses or other notch structures of the syringe assembly mating portion of the medication pump or the like upon rotation. The plurality of angled fin structures and the plurality of protruding structures of the housing are disposed on a neck portion of the housing substantially adjacent to the concentric handle structure of the housing. The plunger member further comprises one or more concentric sealing members disposed about the external periphery thereof configured to seal the liquid medication or the like within the housing. The plunger member further comprises a protruding portion with a sealed tapered end configured to fit inside tube connecting portion of the housing upon full deployment of the plunger member within the housing, thereby ensuring that all medication or the like is expelled. The tube connecting portion of the housing comprises a standard lure connection or the like. The flow restricting collar comprises a tapered portion. Optionally, the flow restricting collar is made of a metal or the like. Preferably, the flow restricting collar has a minimum interior diameter on the order of millimeters or less.

### BRIEF DESCRIPTION OF THE DRAWINGS

The present invention is illustrated and described herein with reference to the various drawings, in which like reference numbers are used to denote like assembly components, and in which:

FIG. 1 is an exploded perspective view of one exemplary embodiment of the syringe assembly of the present invention;

FIG. 2 is another exploded perspective view of one exemplary embodiment of the syringe assembly of the present invention; and

FIG. 3 is an exploded planar view of one exemplary embodiment of the syringe assembly of the present invention.

### DETAILED DESCRIPTION OF THE INVENTION

Again, in various exemplary embodiments, the present invention provides a syringe assembly for a medication pump, such as an insulin pump, or the like that includes a pair of protruding structures at one end that mate with the typical pair of opposed recesses or other notch structures of the syringe assembly mating portion of the pump, without the use of a cap structure and needle member, and that may be manufactured primarily from a relatively inexpensive plastic material or the like, while still providing the medication supply back-pressure required to be adequately sensed by the pump electronics, such that the pump accepts the syringe assembly and is “primed” for proper operation.

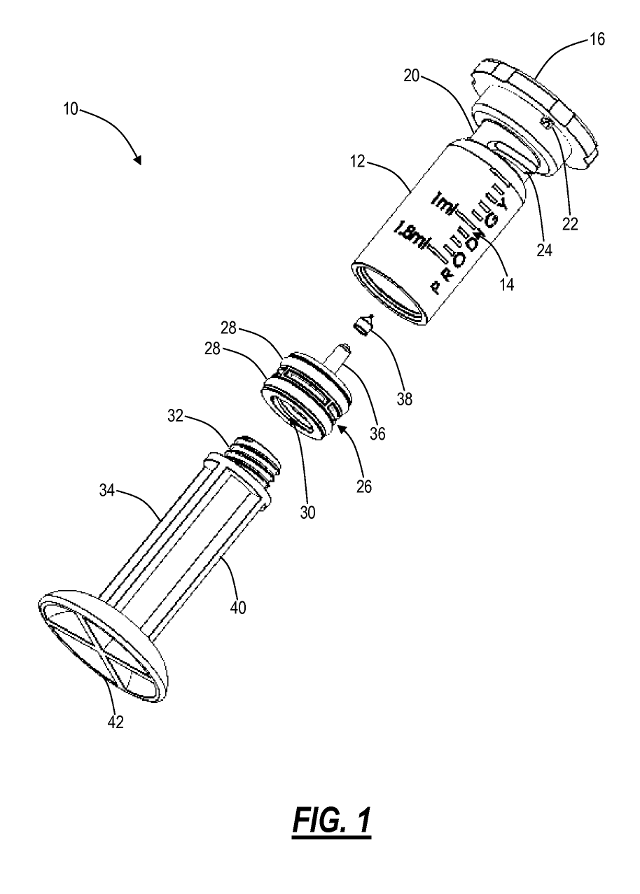

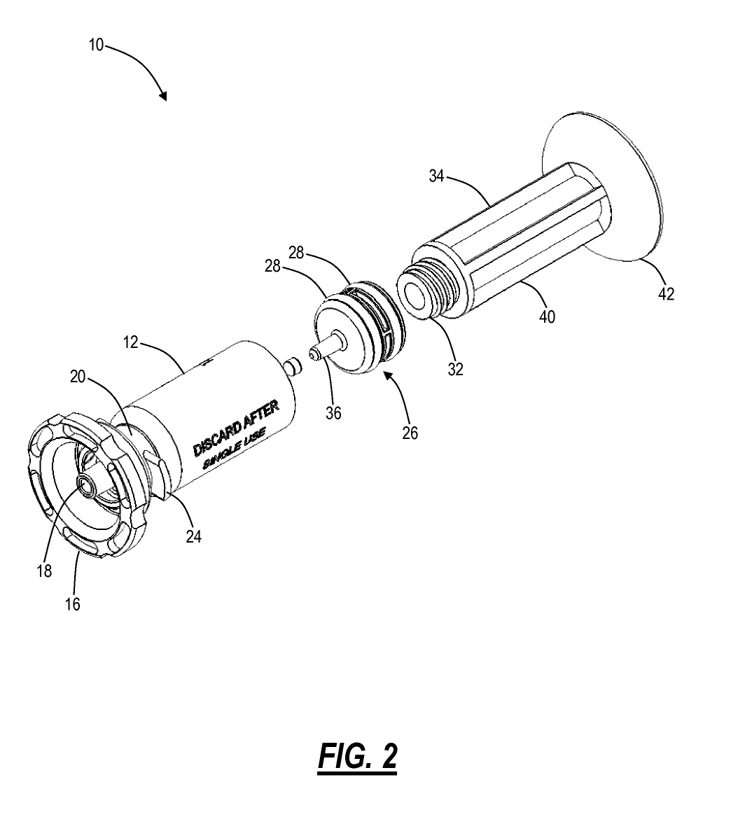

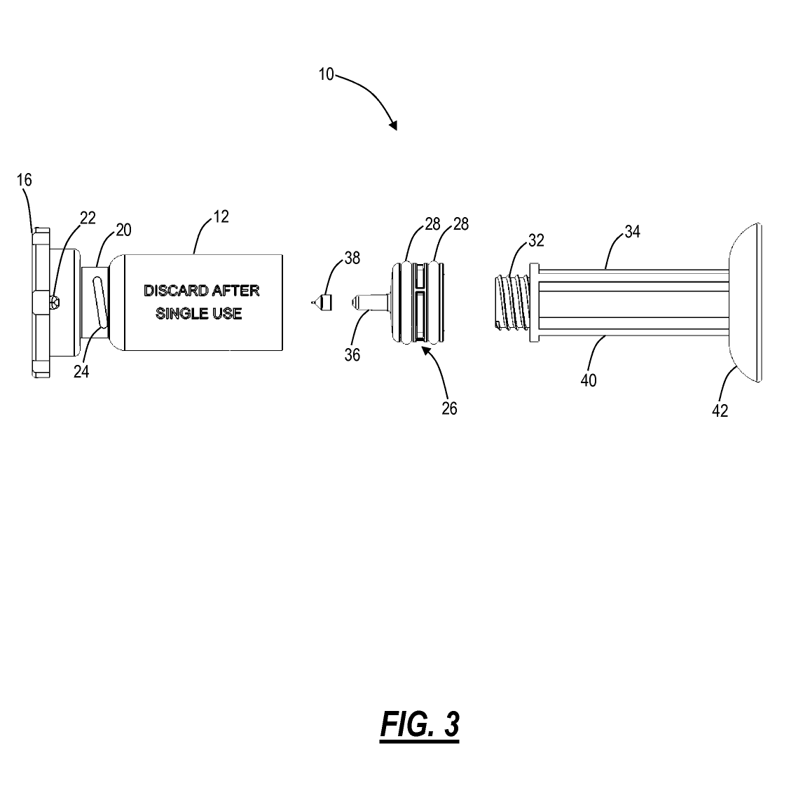

Referring to FIGS. 1-3, in one exemplary embodiment, the syringe assembly 10 includes a substantially cylindrical housing 12 that is designed and configured to contain a predetermined amount of liquid medication or the like. Optionally, the external surface of the housing 12 includes measurement marks 14 for indicating how much liquid medication or the like is contained within the housing 12. As described above, the housing 12 is preferably manufactured from a relatively inexpensive plastic material or the like. The housing 12 has an overall length on the order of tens of millimeters, and a diameter on the order of millimeters. The end portion of the housing 12 includes a concentric handle structure 16 by which the syringe assembly 10 is grasped and handled, such as when it is inserted into a medication pump or the like. A plurality of recessed portions are provided for this purpose. Concentrically disposed within the interior of this handle structure 16 is a conventional protruding tube attachment structure 18 (FIG. 2) to which a tube associated with a medication infusion set or the like is selectively attached once the syringe assembly 10 is inserted into the medication pump or the like. Disposed adjacent to the handle structure, on a neck portion 20 of the housing 12, are a plurality of protruding structures 22 (FIGS. 1 and 3) and a plurality of angled fin structures 24 designed and configured to mate with the typical pair of opposed recesses or other notch structures of the syringe assembly mating portion of the pump, without the use of a cap structure and needle member, as are used conventionally. Specifically, the plurality of fin structures 24 lock the syringe assembly 10 into the pump when the syringe assembly 10 is turned with respect to the pump after insertion via the handle structure 16.

A concentric plunger member 26 is disposed within the housing 12 and is designed and configured to translate along the length of the housing 12 upon actuation, thereby forcing medication or the like from the housing 12. The plunger member 26 has an external diameter on the order of millimeters. In this regard, the plunger member 26 includes one or more concentric sealing o-rings 28 manufactured from compliant rubber or the like disposed in one or more concentric channels manufactured into the exterior of the plunger member 26. This design and configuration essentially seals the medication or the like within the housing 12 when the plunger member 26 is inserted therein, preventing any leakage from about the plunger member 26. One end of the plunger member 26 includes interior threading 30 (FIG. 1) for receiving a corresponding threaded portion 32 of a piston member 34, thereby joining the plunger member 26 to the piston member 34. The other end of the plunger member 26 includes a protruding portion 36 with a sealed tapered end. This protruding portion 36 is designed and configured to fit inside the protruding tube attachment structure 18 of the housing 12 upon full deployment of the plunger member 26 within the housing 12, thereby ensuring that all medication or the like is expelled, as this medication or the like may be very expensive and should not be wasted. In this sense, the protruding portion 36 of the plunger member 26 is substantially conformal with the protruding tube attachment structure 18 of the housing 12. Again, the plunger member 26 and all of its components and the piston member 34 and all of its components may be manufactured from a relatively inexpensive plastic material or the like.

A metallic nozzle collar 38, including a substantially cylindrical portion and a substantially tapering portion, is inserted into an interior opening associated with the protruding tube attachment structure 18 of the housing 12. This nozzle collar 38 has a maximum interior diameter and a minimum interior diameter on the order of fractions of millimeters to millimeters. The nozzle collar 38 is press-fitted into the protruding tube attachment structure 18 of the housing 12, and optionally glued, and is also substantially conformal with the sealed tapered end of the protruding portion 36 of the plunger member 26. This flow restriction structure 38, which is made of a metal or the like, thereby allowing very precise manufacturing tolerances, provides the liquid supply back-pressure required to be adequately sensed by the pump electronics, such that the pump accepts the syringe assembly 10 and is “primed” for proper operation. The use of a plastic material may not provide the required manufacturing tolerances, although it is possible given the state of the manufacturing processes.

The piston member 34, in addition to the threaded portion 32 described above, includes an elongate portion 40 and a wider pump engaging portion 42 that is designed and configured to mate with the drive mechanism of the pump, thereby allowing the piston member 34 to drive the plunger member 26 and selectively expel medication or the like from the housing 12 in a controlled manner. In general, the piston member 34 has an overall length on the order of millimeters.

Although the present invention is illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A syringe assembly for use in a medication pump or the like, comprising:
a housing configured to selectively contain liquid medication or the like;
a plunger member selectively disposed within the housing configured to selectively expel the liquid medication or the like from the housing through a tube connecting portion of the housing; and
a piston member selectively coupled to the plunger member configured to selectively translate the plunger member within the housing;
wherein the housing comprises a concentric handle structure and a plurality of angled fin structures configured to mate with a pair of opposed recesses or other notch structures of a syringe assembly mating portion of the medication pump or the like upon rotation; and
wherein the housing comprises a flow restricting collar disposed therein to limit the flow of the liquid medication or the like from the housing, thereby creating a predetermined back-pressure upon translation of the plunger member within the housing.

2. The syringe assembly of claim 1, wherein the housing further comprises a plurality of protruding structures configured to mate with the pair of opposed recesses or other notch structures of the syringe assembly mating portion of the medication pump or the like upon rotation.

3. The syringe assembly of claim 2, wherein the plurality of angled fin structures and the plurality of protruding structures of the housing are disposed on a neck portion of the housing substantially adjacent to the concentric handle structure of the housing.

4. The syringe assembly of claim 1, wherein the plunger member further comprises one or more concentric sealing members disposed about the external periphery thereof configured to seal the liquid medication or the like within the housing.

5. The syringe assembly of claim 1, wherein the plunger member further comprises a protruding portion with a sealed tapered end configured to fit inside tube connecting portion of the housing upon full deployment of the plunger member within the housing, thereby ensuring that all medication or the like is expelled.

6. The syringe assembly of claim 1, wherein the tube connecting portion of the housing comprises a standard lure connection.

7. The syringe assembly of claim 1, wherein the flow restricting collar comprises a tapered portion.

8. The syringe assembly of claim 1, wherein the flow restricting collar is made of a metal.

9. The syringe assembly of claim 1, wherein the flow restricting collar has a minimum interior diameter on the order of fractions of millimeters to millimeters.

10. A method for providing a syringe assembly for use in a medication pump or the like, comprising:
providing a housing configured to selectively contain liquid medication or the like;
providing a plunger member selectively disposed within the housing configured to selectively expel the liquid medication or the like from the housing through a tube connecting portion of the housing; and
providing a piston member selectively coupled to the plunger member configured to selectively translate the plunger member within the housing;
wherein the housing comprises a concentric handle structure and a plurality of angled fin structures configured to mate with a pair of opposed recesses or other notch structures of a syringe assembly mating portion of the medication pump or the like upon rotation; and
wherein the housing comprises a flow restricting collar disposed therein to limit the flow of the liquid medication or the like from the housing, thereby creating a predetermined back-pressure upon translation of the plunger member within the housing.

11. The method for providing a syringe assembly of claim 10, wherein the housing further comprises a plurality of protruding structures configured to mate with the pair of opposed recesses or other notch structures of the syringe assembly mating portion of the medication pump or the like upon rotation.

12. The method for providing a syringe assembly of claim 11, wherein the plurality of angled fin structures and the plurality of protruding structures of the housing are disposed on a neck portion of the housing substantially adjacent to the concentric handle structure of the housing.

13. The method for providing a syringe assembly of claim 10, wherein the plunger member further comprises one or more concentric sealing members disposed about the external periphery thereof configured to seal the liquid medication or the like within the housing.

14. The method for providing a syringe assembly of claim 10, wherein the plunger member further comprises a protruding portion with a sealed tapered end configured to fit inside tube connecting portion of the housing upon full deployment of the plunger member within the housing, thereby ensuring that all medication or the like is expelled.

15. The method for providing a syringe assembly of claim 10, wherein the tube connecting portion of the housing comprises a standard lure connection.

16. The method for providing a syringe assembly of claim 10, wherein the flow restricting collar comprises a tapered portion.

17. The method for providing a syringe assembly of claim 10, wherein the flow restricting collar is made of a metal.

18. The method for providing a syringe assembly of claim 10, wherein the flow restricting collar has a minimum interior diameter on the order of fractions of millimeters to millimeters.
